(19) United States
(12) Patent Application Publication
(10) Pub. No.: US 2026/0345898 A1
(43) Pub. Date: Nov. 5, 2026

(54) PUMP-TYPE HYDRA NEEDLE FOR PRECISION INFUSION

(71) Applicant: GUANGZHOU VANTEE ELECTRONIC TECHNOLOGY CO. LTD., Guangzhou (CN)

(72) Inventor: Guang YUAN, Guangzhou (CN)

(21) Appl. No.: 19/721,330

(22) Filed: Jun. 26, 2026

(51) Int. Cl. A61M 5/158; A61J 1/20; A61M 5/142; A61M 5/32

(52) U.S. Cl. CPC A61M 5/158; A61J 1/2096; A61M 5/142; A61M 5/3202; A61M 2205/8281

(57) ABSTRACT

A pump-type hydra needle for precision infusion is provided, including a tube body detachably connected to a glass vial. A slidable connection slot and a threaded connection slot that are communicated with each other are formed in the tube body in a direction of being connected to the glass vial. One end of the threaded connection slot is connected to the glass vial, and a large baffle is provided in another end of the threaded connection slot. A through hole is formed in an end portion of the slidable connection slot, and a slidable connection plate is slidably connected to an interior of the through hole. A needle cap mounting slot is formed in the slidable connection plate. A needle assembly is disposed within the needle cap mounting slot, and a needle cap assembly is inserted into the needle cap mounting slot.

### TECHNICAL FIELD

The present disclosure relates to the related field of hydra needles, and specifically, to a pump-type hydra needle for precision infusion.

### BACKGROUND

A current hydra needle on the market is composed of a needle, a needle cap, a flow guide rod, a needle holder, a baffle, and a glass vial. The needle is fixed on the needle holder. The needle cap passes through the needle and is fixed to the needle holder by using a screw. A spring of the flow guide rod is compressed between a telescopic rod and the baffle. The flow guide rod passes through the needle cap, the spring, and needle holder and is connected and fixed to the baffle. The needle holder is detachably connected to the glass vial. During use, since the flow guide rod is positioned higher than the needle passing through the needle cap, the skin is first in contact with the flow guide rod. The flow guide rod then drives the baffle to move backward. Liquid components in the glass vial flow out through a gap between the telescopic rod and the needle cap or the needle holder, and at the same time, the needle pierces into the skin to achieve infusion. This structural design has the following shortcomings and deficiencies:
1. Since the liquid components can only flow out through the gap between the telescopic rod and the needle cap/needle holder, this results in non-uniform liquid discharge or even a failure to discharge liquid.
2. The liquid can only be discharged through a gap around the flow guide rod. No liquid is delivered synchronously with each needle when each needle pierces into the skin. Moreover, the liquid is in constant contact with the flow guide rod and the spring, triggering a chemical reaction between the spring and the liquid, which contaminates the liquid and makes cleaning extremely difficult.
3. The force with which the needle pierces into the skin cannot be controlled.

### SUMMARY

The present disclosure aims to provide a pump-type hydra needle for precision infusion, to solve the problems mentioned in the foregoing background section.

In order to achieve the above objective, the present disclosure provides the following technical solutions:

A pump-type hydra needle for precision infusion includes a tube body detachably connected to a glass vial. A slidable connection slot and a threaded connection slot that are communicated with each other are formed in the tube body in a direction of being connected to the glass vial, and inner diameters of the slidable connection slot and the threaded connection slot gradually decrease. One end of the threaded connection slot is connected to the glass vial, and a large baffle is provided in another end of the threaded connection slot. A step between the large baffle and the threaded connection slot and a step between the large baffle and the slidable connection slot are in snap-fit. A through hole is formed in an end portion of the slidable connection slot, and a slidable connection plate is slidably connected to an interior of the through hole. A diameter of a top of the through hole is less than a diameter of the slidable connection plate. An inner diameter of the slidable connection slot is greater than the diameter of the slidable connection plate. A needle cap mounting slot is formed in the slidable connection plate. A needle assembly is disposed within the needle cap mounting slot, and a needle cap assembly is inserted into the needle cap mounting slot. The needle cap assembly is disposed at a periphery of the needle assembly and is spaced apart from the slidable connection plate. A flow guide rod that is of a hollow structure is further inserted into the slidable connection plate. The flow guide rod is inserted into the large baffle and is slidably connected to the large baffle. An end portion of the flow guide rod is mounted in a flow guide rod mounting slot of a small baffle, and two U-shaped holes are symmetrically formed in the flow guide rod. A height of each U-shaped hole is less than a thickness of the large baffle. A spring with two ends respectively connected to the large baffle and the slidable connection plate is further sleeved at a periphery of the flow guide rod. When a clearance exists between the small baffle and the large baffle, the threaded connection slot is communicated with a spacing between the slidable connection plate and the needle cap assembly.

In a further solution of the present disclosure, the needle assembly includes a plurality of needle bodies and needle tips. The plurality of needle bodies are mounted on the slidable connection plate and are equidistantly distributed in a circumferential direction of the slidable connection plate. The needle tips are arranged at end portions of the plurality of needle bodies.

In a further solution of the present disclosure, the needle cap assembly includes a needle cap plate and a needle cap mounting plate. One end of the needle cap mounting plate is inserted into the needle cap mounting slot, and another end is connected to the needle cap plate. The needle cap plate is slidably connected to an inner wall of the slidable connection slot. The needle cap plate is spaced apart from the slidable connection plate, and an outflow hole is provided at a spacing therebetween. A plurality of through holes are formed in both the needle cap plate and the needle cap mounting plate, and positions of the through holes correspond to the needle bodies. Clearances exist between the through holes and the needle bodies.

In a further solution of the present disclosure, four limiting posts are further symmetrically arranged on two sides of the slidable connection plate close to the threaded connection slot. Clearances exist between the four limiting posts and the large baffle. The four limiting posts are slidably connected to an inner wall of the slidable connection slot.

In a further solution of the present disclosure, an outer wall of the slidable connection slot is further detachably sleeved with a needle body cap.

Compared with the prior art, the present disclosure has the beneficial effects:
1. The two ends of the flow guide rod that is of the hollow structure respectively communicate an overflow clearance between the threaded connection slot and the slidable connection plate with an overflow clearance between the threaded connection slot and the needle cap plate, and the overflow clearances are communicated with clearances between the needle cap plate and the needle bodies to restrict the liquid to only pass through the flow guide rod and not be in contact with the spring, so that the liquid infiltrates the skin along the needles to achieve precision infusion. Furthermore, the liquid can flow out through the clearances between the needle bodies and the needle cap plate, so that the liquid can be discharged from every needle hole, thus achieving an efficient and uniform liquid discharging effect. Since the liquid is not in contact with the spring, problems of liquid accumulation and contamination are avoided. Moreover, since the clearances between the needle cap plate and the needle bodies are communicated with the entire flow guide rod, it is very convenient for cleaning. Compared with the conventional hydra needle, the present disclosure solves the following problems: The liquid in the conventional hydra needle can only be discharged through a gap around the telescopic rod. Moreover, no liquid is delivered synchronously with each needle when each needle pierces into the skin, resulting in intermittent liquid delivery and non-uniform liquid discharge. In addition, the liquid is in constant contact with the telescopic rod and the spring, thus triggering a chemical reaction between the spring and the liquid, which compromises the liquid efficacy and makes overall cleaning extremely difficult.
2. By setting a distance between end portions of the limiting posts and the large baffle, under the elastic force of the spring, due to the existence of the limiting post, at the maximum displacement, all the needle bodies pierce the skin, so that the force with which the needle bodies pierce the skin can be adjusted, and the safety of operation is enhanced. With the foregoing design, the force with which the needle bodies pierce into the skin and the force with which the needle bodies are withdrawn from the skin are gentle and applied gradually, thereby reducing the user's pain during operation. This solves the problems of inconsistent piercing depths of the needles, excessive pain, and complete reliance on the operator's experience during operation because the force with which the conventional hydra needle pierces into the skin cannot be controlled.

### BRIEF DESCRIPTION OF THE DRAWINGS

FIG. 1 is a schematic diagram of a structure of an unfolded state according to the present disclosure.

FIG. 2 is a schematic diagram of a structure of a combined state according to the present disclosure.

FIG. 3 is a schematic diagram of a structure of a position of an outflow hole formed between a slidable connection plate and a needle cap plate according to the present disclosure.

FIG. 4 is a front view of the present disclosure.

Numerals in the accompanying drawings: 1: small baffle; 2: flow guide rod mounting slot; 3: large baffle; 4: threaded connection slot; 5: slidable connection slot; 6: through hole; 7: U-shaped hole; 8: slidable connection plate; 9: needle cap mounting slot; 10: needle body; 11: needle cap plate; 12: needle cap mounting plate; 13: needle tip; 14: limiting post; 15: spring; 16: tube body; 17: flow guide rod; 19: outflow hole; and 18: needle body cap.

### DETAILED DESCRIPTION OF THE EMBODIMENTS

The following embodiments will describe the present disclosure in detail with reference to the accompanying drawings. In the accompanying drawings and this specification, identical or similar parts are denoted by the same reference numerals. In addition, in practical applications, the shape, thickness, and height of the components may be enlarged or reduced. The embodiments listed in the present disclosure are merely illustrative of the present disclosure and shall not be construed as limiting the scope of the present disclosure. Any apparent modifications or changes made to the present disclosure shall not depart from the spirit and scope of the present disclosure.

### Embodiment 1

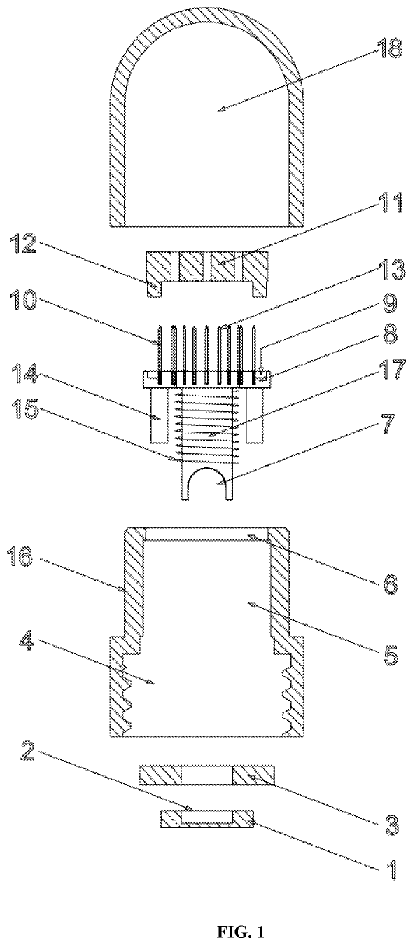

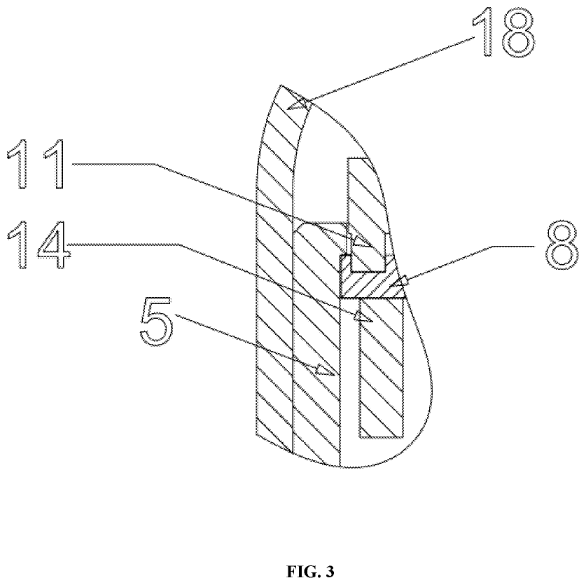

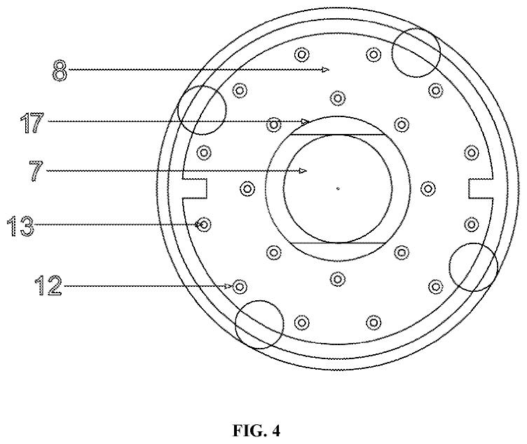

Referring to FIG. 1 to FIG. 4, in this embodiment, a pump-type hydra needle for precision infusion includes a tube body 16 detachably connected to a glass vial. A slidable connection slot 5 and a threaded connection slot 4 that are communicated with each other are formed in the tube body 16 in a direction of being connected to the glass vial, and inner diameters of the slidable connection slot 5 and the threaded connection slot 4 gradually decrease. One end of the threaded connection slot 4 is connected to the glass vial, and a large baffle 3 is provided in another end of the threaded connection slot. A step between the large baffle 3 and the threaded connection slot 4 and a step between the large baffle 3 and the slidable connection slot 5 are in snap-fit. A through hole 6 is formed in an end portion of the slidable connection slot 5, and a slidable connection plate 8 is slidably connected to an interior of the through hole 6. A diameter of a top of the through hole 6 is less than a diameter of the slidable connection plate. An inner diameter of the slidable connection slot 5 is greater than the diameter of the slidable connection plate 8, thus restricting the slidable connection plate 8 to only slide inside the slidable connection slot 5 and the through hole 6. A needle cap mounting slot 9 is formed in the slidable connection plate 8. A needle assembly is disposed within the needle cap mounting slot 9, and a needle cap assembly is inserted into the needle cap mounting slot. The needle cap assembly is disposed at a periphery of the needle assembly and is spaced apart from the slidable connection plate 8. A flow guide rod 17 that is of a hollow structure is further inserted into the slidable connection plate 8. The flow guide rod 17 is inserted into the large baffle 3 and is slidably connected to the large baffle 3. An end portion of the flow guide rod 17 is mounted in a flow guide rod mounting slot 2 of a small baffle 1, and two U-shaped holes 7 are symmetrically formed in the flow guide rod 17. A height of each U-shaped hole 7 is less than a thickness of the large baffle 3. A spring 15 with two ends respectively connected to the large baffle 3 and the slidable connection plate 8 is further sleeved at a periphery of the flow guide rod 17. When a clearance exists between the small baffle 1 and the large baffle 3, the threaded connection slot 4 is communicated with a spacing between the slidable connection plate 8 and the needle cap assembly. Thus, the liquid in the glass vial enters the flow guide rod 17 through the U-shaped holes 7, flows into a clearance between the slidable connection plate 8 and a needle cap plate 11, and flows toward needle tips 13 synchronously with needle bodies 10, so that the liquid is delivered into the skin.

In this embodiment, the needle assembly includes a plurality of needle bodies 10 and needle tips 13. The plurality of needle bodies 10 are mounted on the slidable connection plate 8 and are equidistantly distributed in a circumferential direction of the slidable connection plate 8. The needle tips 13 are arranged at end portions of the plurality of needle bodies 10.

In this embodiment, the needle cap assembly includes a needle cap plate 11 and a needle cap mounting plate 12. One end of the needle cap mounting plate 12 is inserted into the needle cap mounting slot 9, and another end is connected to the needle cap plate 11. The needle cap plate 11 is slidably connected to an inner wall of the slidable connection slot 5. The needle cap plate 11 is spaced apart from the slidable connection plate 8, and an outflow hole 19 is provided at a spacing therebetween. A plurality of through holes are formed in both the needle cap plate 11 and the needle cap mounting plate 12, and positions of the through holes correspond to the needle bodies 10. Clearances exist between the through holes and the needle bodies 10.

The working principle is as follows: When the needle tips 13 are in contact with the skin, a treatment head (the slidable connection plate 8, the needle cap plate 11, and the needle bodies 10) as an integral part is pushed to drive the flow guide rod 17 to move toward the threaded connection slot 4 and compress the spring 15 at the same time. An end portion of the flow guide rod 17 drives the small baffle 1 to move away from the large baffle 3. Based on the principle of pumping water, the liquid flows into the flow guide rod 17 through the U-shaped holes 7, and then flows to the outflow hole 19 in the spacing between the slidable connection plate 8 and the needle cap mounting plate 12 along with the flow guide rod 17. Since the spring 15 is compressed to generate resistance, the needle bodies 10 are driven to move toward the skin and are pushed to slowly pierce into the skin. When the four limiting posts 14 reach their maximum displacements, parts of the needle bodies 10 exposed out of the needle cap mounting plate 12 all pierce into the skin, and then the liquid will flow out through the clearances between the needle bodies 10 and the through holes of the needle cap mounting plate 12 and flow into the skin synchronously with the needle bodies 10.

When an external force pulls the needle bodies out of the skin, the flow guide rod 17 can drive the small baffle 1 to move toward the large baffle 3 under the elastic force of the spring 15. In the approaching process, the small baffle 1 can push the liquid around to enter the flow guide rod 17 through the U-shaped holes 7, to prepare for next liquid discharge. Finally, when the elastic force reaches its minimum, the U-shaped holes 7 retract into the large baffle 3, and sealing is completed through the small baffle 1. Meanwhile, due to the factor of the elastic force of the spring 15, the needle bodies 10 piercing into the skin can be automatically pulled out of the skin relatively slowly under the elastic force, without applying an external force.

In summary, the two ends of the flow guide rod 17 that is of the hollow structure respectively communicate the large baffle 3 fixed by the threaded connection slot 4 with the outflow hole 19 that spaces the slidable connection plate 8 and the needle cap plate 11, so that the present disclosure solves the following problems: The liquid in the conventional hydra needle can only be discharged through a gap around the flow guide rod 17. Moreover, no liquid is delivered synchronously with each needle when each needle body 10 pierces into the skin. In addition, the liquid is in constant contact with the flow guide rod 17 and the spring 15, thus triggering a chemical reaction between the spring 15 and the liquid, which compromises the liquid efficacy and makes overall cleaning extremely difficult.

### Embodiment 2

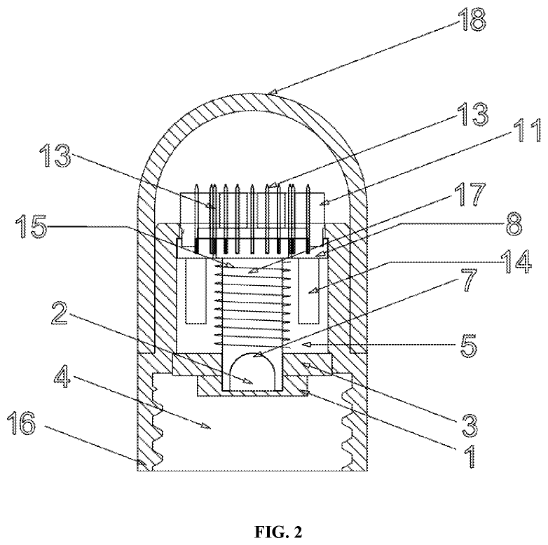

Referring to FIG. 1 to FIG. 2, in this embodiment, four limiting posts 14 are further symmetrically arranged on two sides of the slidable connection plate 8 close to the threaded connection slot 4. Clearances exist between the four limiting posts 14 and the large baffle 3. The four limiting posts 14 are slidably connected to an inner wall of the slidable connection slot 5.

When the limiting posts 14 move toward the large baffle 3 and reach their maximum displacements, the limiting posts 14 are in contact with the large baffle 3. In this case, the needle bodies 10 also reach their maximum motion displacements within the slidable connection slot 5. Then, under the resistance of the spring 15, the needle bodies 10 and the needle tips 13 are driven to slowly pierce into the skin. At their maximum displacements, all the needle bodies 10 pierce into the skin. By setting a distance between end portions of the limiting posts 14 and the large baffle 3, piercing depths of the needle bodies 10 can be sensed, thus solving the problems of inconsistent piercing depths of the needles and complete reliance on the operator's experience during operation because the force with which the conventional hydra needle pierces into the skin cannot be controlled.

### Embodiment 3

Referring to FIG. 1 to FIG. 2, in this embodiment, an outer wall of the slidable connection slot 5 is further detachably sleeved with a needle body cap 18.

For storage, the needle body cap 18 is closed, thus enhancing the overall sealing property while protecting the needle tips 13.

Standard parts used in the present disclosure can be purchased in the market, and specially-shaped parts can be customized according to the instructions of this specification and the accompanying drawings. The specific connection manners of the parts are mature conventional manners such as medical ultraviolet (UV) shadowless adhesive fixation in the prior art. Tools and equipment that are required in an assembly process all use conventional model numbers in the prior art. These will not be elaborated herein.

For those skilled in the art, it is apparent that the present disclosure is not limited to the details of the exemplary embodiments mentioned above, and can be implemented in other specific forms without departing from the spirit or basic features of the present disclosure. Therefore, in any perspective, the embodiments should be regarded as exemplary and non-restrictive. The scope of the present disclosure is limited by the accompanying claims rather than the above description. Therefore, all changes within the meaning and scope of the equivalent conditions of the claims within the present disclosure.

In addition, it should be understood that although this specification is described according to the implementations, not each implementation only includes one independent technical solution. This narration method of this specification is only for clarity. A person skilled in the art should regard this specification as a whole, and the technical solutions in the respective embodiments can also be appropriately combined to form other implementations that can be understood by a person skilled in the art.

What is claimed is:

1. A pump-type hydra needle for precision infusion, comprising a tube body (16) detachably connected to a glass vial, wherein a slidable connection slot (5) and a threaded connection slot (4) that are communicated with each other are formed in the tube body (16) in a direction of being connected to the glass vial, and inner diameters of the slidable connection slot (5) and the threaded connection slot (4) gradually decrease; one end of the threaded connection slot (4) is connected to the glass vial, and a large baffle (3) is provided in another end of the threaded connection slot (4); a step between the large baffle (3) and the threaded connection slot (4) and a step between the large baffle (3) and the slidable connection slot (5) are in snap-fit; a through hole (6) is formed in an end portion of the slidable connection slot (5), and a slidable connection plate (8) is slidably connected to an interior of the through hole (6); a diameter of a top of the through hole (6) is less than a diameter of the slidable connection plate (8); an inner diameter of the slidable connection slot (5) is greater than the diameter of the slidable connection plate (8); a needle cap mounting slot (9) is formed in the slidable connection plate (8); a needle assembly is disposed within the needle cap mounting slot (9), and a needle cap assembly is inserted into the needle cap mounting slot (9); the needle cap assembly is disposed at a periphery of the needle assembly and is spaced apart from the slidable connection plate (8); a flow guide rod (17) that is of a hollow structure is further inserted into the slidable connection plate (8); the flow guide rod (17) is inserted into the large baffle (3) and is slidably connected to the large baffle (3); an end portion of the flow guide rod (17) is mounted in a flow guide rod mounting slot (2) of a small baffle (1), and two U-shaped holes (7) are symmetrically formed in the flow guide rod (17); a height of each U-shaped hole (7) is less than a thickness of the large baffle (3); a spring (15) with two ends respectively connected to the large baffle (3) and the slidable connection plate (8) is further sleeved at a periphery of the flow guide rod (17); and when a clearance exists between the small baffle (1) and the large baffle (3), the threaded connection slot (4) is communicated with a space between the slidable connection plate (8) and the needle cap assembly.

2. The pump-type hydra needle for precision infusion according to claim 1, wherein the needle assembly comprises a plurality of needle bodies (10) and needle tips (13); the plurality of needle bodies (10) are mounted on the slidable connection plate (8) and are equidistantly distributed in a circumferential direction of the slidable connection plate (8); and the needle tips (13) are arranged at end portions of the plurality of needle bodies (10).

3. The pump-type hydra needle for precision infusion according to claim 1, wherein the needle cap assembly comprises a needle cap plate (11) and a needle cap mounting plate (12);
one end of the needle cap mounting plate (12) is inserted into the needle cap mounting slot (9), and another end is connected to the needle cap plate (11); the needle cap plate (11) is slidably connected to an inner wall of the slidable connection slot (5); the needle cap plate (11) is spaced apart from the slidable connection plate (8), and an outflow hole (19) is provided at a spacing therebetween; a plurality of through holes are formed in both the needle cap plate (11) and the needle cap mounting plate (12), and positions of the through holes correspond to the needle bodies (10); and clearances exist between the through holes and the needle bodies (10).

4. The pump-type hydra needle for precision infusion according to claim 2, wherein four limiting posts (14) are further symmetrically arranged on two sides of the slidable connection plate (8) close to the threaded connection slot (4); clearances exist between the four limiting posts (14) and the large baffle (3); and the four limiting posts (14) are slidably connected to an inner wall of the slidable connection slot (5).

5. The pump-type hydra needle for precision infusion according to claim 2, wherein an outer wall of the slidable connection slot (5) is further detachably sleeved with a needle body cap (18).
